(19) United States
(12) Patent Application Publication
(10) Pub. No.: US 2006/0074487 A1
(43) Pub. Date: Apr. 6, 2006

(54) Intraocular device for the restoring visual accommodation of presbiopic eye

(76) Inventor: Alain-Nicolas Gilg, Lyon (FR)

Correspondence Address: YOUNG & THOMPSON, 745 SOUTH 23RD STREET, 2ND FLOOR, ARLINGTON, VA, 22202, US

(21) Appl. No.: 10/529,232

(22) PCT Filed: Sep. 24, 2003
(86) PCT No.: PCT/FR03/02812

§ 371 Date: Mar. 25, 2005

(30) Foreign Application Priority Data
Sep. 25, 2002 (FR) 02/11870

(51) Int. Cl. A61F 2/14; A61F 9/013

(52) U.S. Cl. 623/4.1

(57) ABSTRACT

A device for treating an eye affection related to a decrease of ocular accommodation amplitude resulting from loss of tension of the zonular fibers caused by increase in the lens diameter. The device is designed to be surgically implanted in the ciliary sulcus, behind the iris, and configured to compensate the loss of tension of the zonular fibers by exerting on the ciliary sulcus a pressure tending to increase its diameter. The device includes a closed tubular envelope made of an elastic and fluid-impermeable material, the envelope being shaped like a ring or a ring portion having a predetermined outer diameter at rest, the inner ring wall of the envelope delimiting a lumen adapted to be filled with an incompressible fluid tending to increase the outer diameter up to a value whereby the loss of tension of the zonular fibers is compensated.

The present invention relates to the field of treatment of ocular conditions associated with a diminution of the amplitude of accommodation of the eye, in particular presbyopia.

The ocular conditions leading to a diminution of the amplitude of accommodation of the eye such as presbyopia are due principally to a loss of tension of the zonular fibres, linked to age, and associated with an increase in diameter of the lens, secondary atrophy of the ciliary muscle, loss of efficiency in the movements of the vitreous humour.

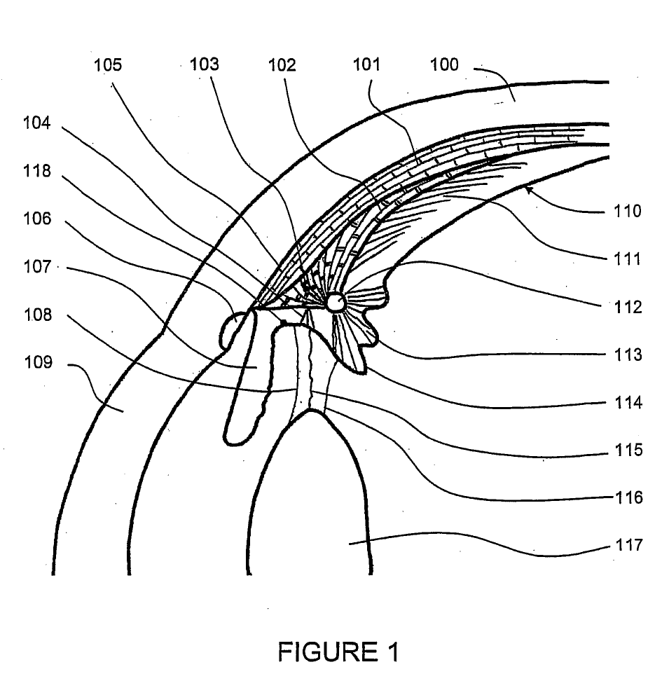

FIG. 1 shows an upper sagittal semi-section of the human eye at rest, without accommodation. On this figure are shown respectively the sclera (100), the posterior longitudinal ciliary muscle (101), the posterior radial ciliary muscle (102), the anterior radial ciliary muscle (103), the anterior longitudinal ciliary muscle (105), the first bundle of ciliary collagen fibres (104), the trabecular meshwork (106), the iris (107), the anterior zonule (108), the cornea (109), the pars plana (110), the collagen fibres linked to the pars plana (111), the circular ciliary muscle (112), the 2^{nd} bundle of ciliary collagen fibres (113), the ciliary processes (114), the equatorial zonule (115), the posterior zonule (116) and the non-accommodated lens (117).

During accommodation, the circular ciliary muscle (112) is displaced in the direction of the lens (117), the anterior radial ciliary muscle (103) is displaced towards the sclera (100). The combined action of these two muscles results in the placing of the equatorial zonule under tension (115) through the intermediary of the first bundle of collagen fibres (104) and in a relaxation of the posterior zonule (116) by displacement and tilting of the ciliary processes (114) in the direction of the lens (117) through the intermediary of the 2^{nd} bundle of collagen fibres (113).

Thus, in a non-presbyopic eye in the course of accommodation, the equatorial zonule (115) transmits the forces of the anterior radial ciliary muscle (103), and this causes an increase of the equatorial diameter of the lens. The posterior (116) and anterior (108) zonules maintain the lens in its position during accommodation. During ciliary contraction, through the intermediary of the longitudinal (105 and 101) and radial (103 and 102) ciliary muscles, the equatorial zonule (115) is placed under tension and simultaneously the anterior (108) and posterior (116) zonules are relaxed. The presence of the collagen fibres (104) connecting the anterior part of the radial muscle (103) to the ciliary sulcus (118) and the presence of the collagen fibres (113) between the circular ciliary muscle (112) and the ciliary processes (114) account for the fact that the accommodation is accompanied by a placing of the equatorial zonule under tension (115) and by a concomitant relaxation of the posterior fibres (116).

It emerges from the foregoing that during accommodation, the contraction of the ciliary muscle causes a diminution of the distance between the equator of the lens (117) and the sclera (100) leading to a convexity of the central part of the lens and an increase of its antero-posterior diameter.

With age, the continued growth of the lens associated with a sclera without the possibility of growth has the effect of leading to a diminution of the effective working distance of the ciliary musculature and hence to a loss of the tension capacity of the zonule fibres.

In order to restore the zonules to a state of tension, it has been suggested that an annulus capable of being applied against the ciliary sulcus (118) be placed within the eye in order to obtain a displacement of the anchorage zone from the equatorial zonule and to restore a state of tension.

The French patent application published under the No. 2.787.991 describes such a device which is constituted of an annulus exhibiting an axis of rotation and a first and second margin displaced towards the radial directions and towards the direction of the said axis of rotation, the first margin being designed to provide support through at least a part of its length, over a part of the internal wall of the eye and the said second margin being capable of being applied against a median zone of the zonules of the lens. Thus, the device described is maintained in place at the level of the sulcus by its external margin, the internal margin, displaced from the external margin in a radial direction, providing support on the zonules in order to restore them to a state of tension.

According to a first embodiment, the tension annulus is made of a flexible biocompatible material in order to make possible its insertion within the eye.

According to a second embodiment, the annulus is open and is made of a rigid biocompatible material.

A tension annulus such as that described in the French patent application No. 2.787.991 is such as to make possible a restoration of the zonules under tension and thus reestablish at least partially the accommodation capacities of the eye.

Such a tension annulus, however, exhibits many disadvantages which are itemized below.

First, when the annulus is made of a rigid material, it can not, by definition, be introduced into the eye after preliminary bending, as is usually the case for the introduction of intra-ocular devices. The introduction of the rigid, unbent annulus requires a large incision in the cornea prior to its introduction or necessitates potentially traumatic operations for its introduction.

Irrespective of the embodiment considered, such a tension annulus, which possesses a predefined geometry when it is manufactured, possesses an invariable intrinsic elasticity effect as a result of its fixed diameter.

Now, it has been observed that in mammals and particularly in man, the diameter of the sulcus varies from one individual to another.

In order to treat presbyopia in all patients with a tension annulus such as described in the French patent application No. FR 2.787.991, it would thus be necessary to have available a series of annuli of different diameters adapted to the diameter of the sulcus of each patient. Now the anatomical localisation of the sulcus which forms a groove between the ciliary body and the iris, makes the in vivo determination of its diameter technically difficult, prior to a surgical operation. Consequently, the choice of a tension annulus such as described in the application FR 2 787 991 of a diameter specifically adapted to the patient is dependent on an empirical assessment.

Another disadvantage of the tension annulus known from the state of the art is related to the fact that the diminution of the amplitude of accommodation of the eye is not a sudden phenomenon but, on the contrary, a continuous phenomenon, which increases with ageing over the course of time.

It is considered that in the case of presbyopia the diameter of the lens increases by about 20 μm per year over a period of 20 years, usually from the age of 40 to 60 years. During ageing the diameter of the lens thus increases by about 400 μm.

In order to maintain an optimal correction during the course of time, it would thus be necessary to regularly replace the implanted tension armulus by an annulus of greater diameter capable of compensating for the increasing loss of tension of the zonules.

The applicant decided to develop an improved device which resolves the technical disadvantages of the previous devices. The applicant has in particular taken into account the fact that the mechanical constraint making it possible to compensate the loss of tension of the zonular fibres varies with time and that an optimal correction would necessitate recourse to a device which would make it possible to vary the mechanical constraints causing a restoration of the tension of the zonular fibre in a continuous manner as the diameter of the lens increases.

To the knowledge of the applicant, no device of the state of the art makes possible such a correction of the amplitude of accommodation of the eye in a continuous manner as the ocular condition, in particular presbyopia, worsens.

The technical disadvantages of the previous devices described above have been resolved according to the invention.

The applicant decided to develop a device for treating an ocular condition associated with a diminution of the amplitude of accommodation which would make it possible to treat the entire population of patients, irrespective of the diameter of the ciliary sulcus and however advanced the condition, in particular irrespective of the extent of progression of the presbyopia.

It has been shown according to the invention that a device available in the form of an annulus, open or closed and the external diameter of which can be varied, makes it possible, once introduced into the eye at the level of the sulcus, to restore the tension of the zonular fibres to a sufficient extent to compensate solely for the loss of tension caused by the increase of the diameter of the lens in a given patient at a given moment in the course of the development of his ocular condition.

The object of the present invention is a device for treating an ocular condition linked to a diminution of the amplitude of accommodation of the eye due to a loss of tension of the zonular fibres caused by an increase of the diameter of the lens, said device being designed to be implanted surgically in the ciliary sulcus, behind the iris, and defined so as to compensate the loss of tension of the zonular fibres by exerting on the ciliary sulcus a pressure tending to increase its diameter, characterized in that the said device comprises a closed tubular envelope (1) consisting of an elastic material impermeable to fluids, the envelope (1) having the form of an annulus or of a portion of an annulus having an external diameter (20) predetermined at rest, the internal wall (11) of the envelope (1) defining an aperture (12) designed to be filled with an incompressible fluid tending to increase the external diameter (20) to a value at which the compensation of the loss of tension of the zonular fibres is obtained.

Other characteristics and advantages of the invention will become more apparent on reading the description that follows of the embodiment of the invention given by non-limiting examples. The description relates to the appended Figures on which:

FIG. 1 shows an upper sagittal semi-section of a human eye at rest without accommodation;

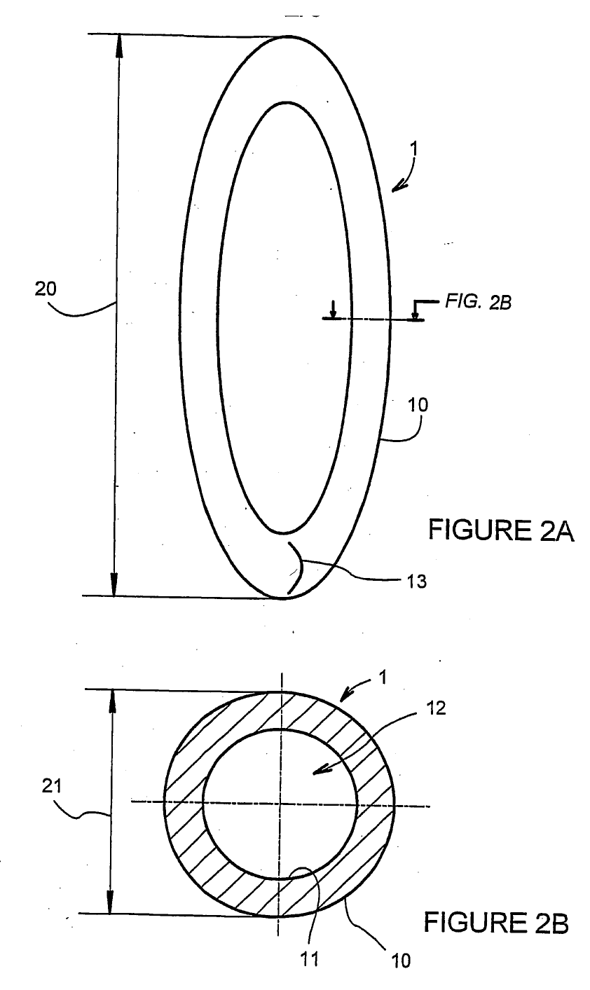

FIG. 2 is a drawing of the device according to the invention having the form of a closed annulus.

FIG. 2A shows the entire device.

FIG. 23 shows a section of the closed tubular envelope (1).

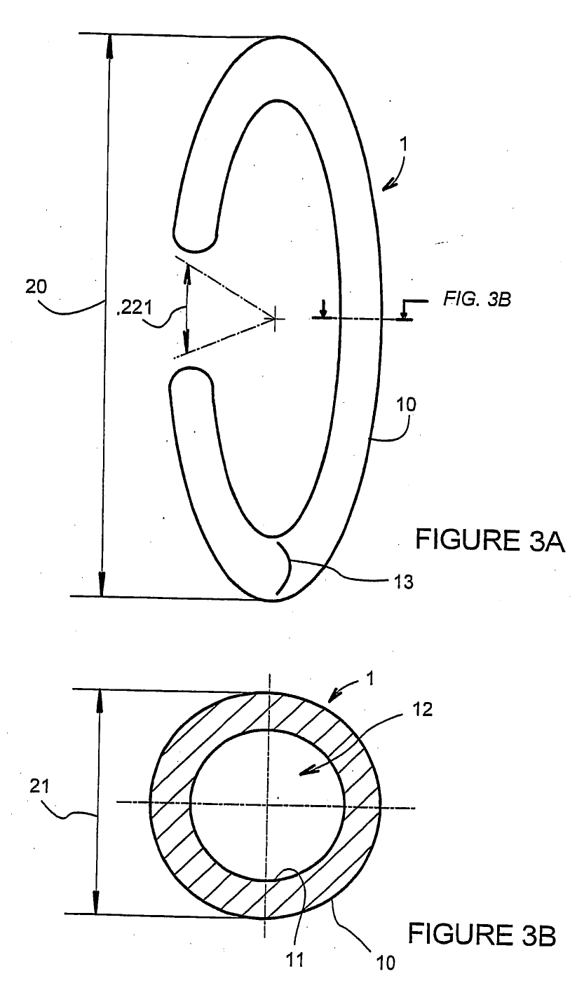

FIG. 3 shows a drawing of the embodiment of the device according to the invention in which the closed tubular envelope (1) has the form of a portion of an annulus and constitutes an open annulus; FIG. 3A shows the complete device; FIG. 3B shows a section of the closed tubular envelope (1);

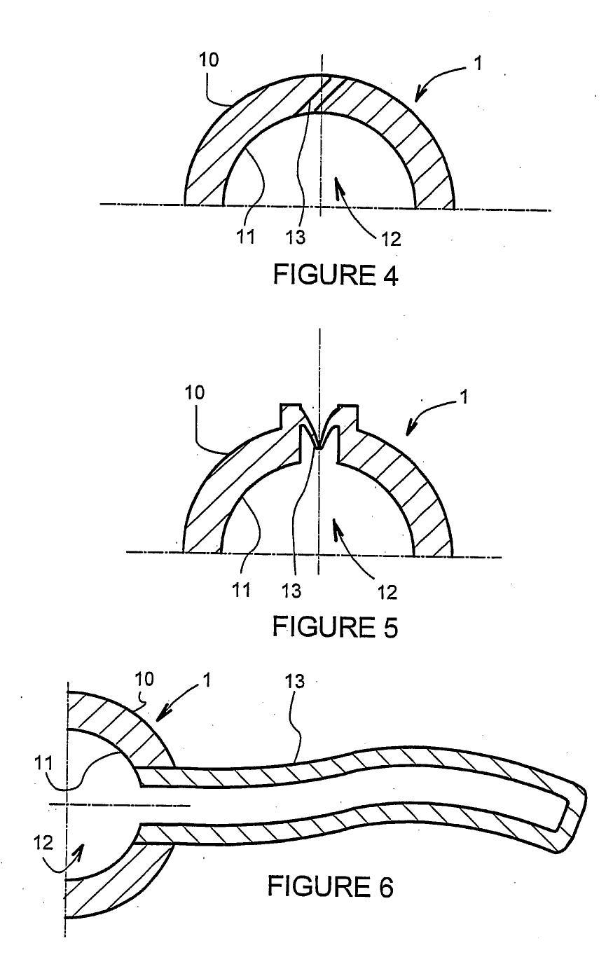

FIG. 4 is a first embodiment of a valve (13) constituted by a partial section of the envelope (1) with a bevelled edge;

FIG. 5 shows a second embodiment of a valve (13) comprising a mobile shutter of the “Duckbill valve” type.

FIG. 6 shows a third embodiment of a valve (13) in which the envelope (1) is equipped with a catheter type tube (13) comprising a valve with its extremity opposite the aperture (12) of the envelope (1);

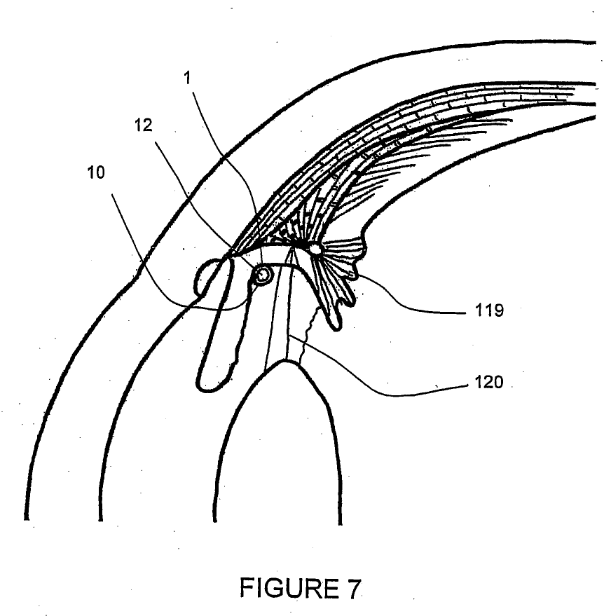

FIG. 7 is a drawing of an upper-sagittal semi-section of an eye, in which the device according to the invention was introduced into the ciliary sulcus.

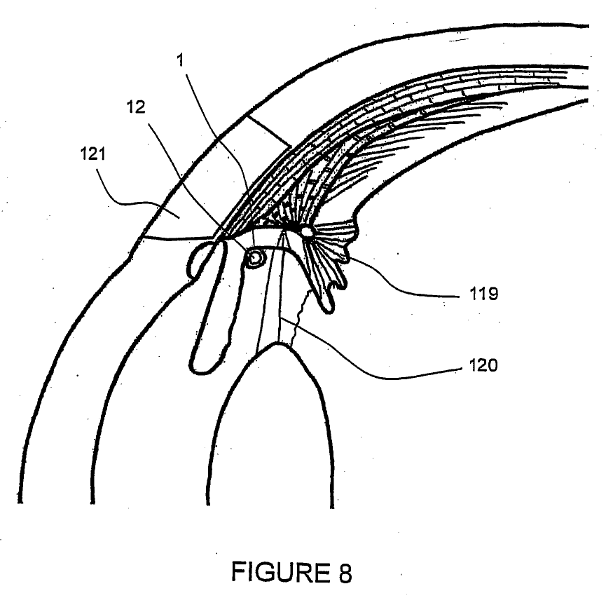

FIG. 8 shows an upper sagittal semi-section of an eye at rest without accommodation, with the device according to the invention in place in the ciliary sulcus, the eye having undergone an anterior ciliary sclerotomy opposite (121).

The envelope (1) is constituted of a biocompatible material, the mechanical characteristcs of which confer on it sufficient rigidity to preserve its annular form when the aperture (12) is filled with an incompressible fluid. Furthermore, the material of which the envelope (1) is constituted must be sufficiently flexible or elastic to expand and thus increase the external diameter (20) of the device after the aperture (12) has been filled with the incompressible fluid at a pressure value of fluid greater than the pressure for which the annulus or the portion of the annulus is considered to be “at rest”.

The envelope (1) is “at rest” when the aperture (12) is completely filled with the incompressible fluid without causing expansion of the envelope (1).

The determination of the stage at which the envelope (1) is “at rest” may be easily achieved by the specialist skilled in the art. As an illustration, the specialist skilled in the art may use a filling device equipped with a means for the measurement of the pressure of the incompressible fluid in the aperture (12) of the envelope (1), for example a micromanometer, placed between the filling device and the entrance of the aperture (12). At the start of the filling of the envelope (1), if the latter is done at constant flow rate, the pressure measured is itself approximately constant Starting from a value of the volume of incompressible fluid introduced into the aperture (12) of the envelope (1), an increase of the pressure is observed. The pressure increases when the volume of the aperture (12) is completely filled with the incompressible fluid and when any additional volume of incompressible fluid introduced into the aperture (12) causes an expansion of the walls of the envelope (1).

Preferably, the envelope (1) is constituted of a biocompatible elastomeric material.

By “elastomeric material” according to the invention is meant a material which can be extended repeatedly up to twice its initial length (200% of the initial length) at physiological temperature (37° C.) and which rapidly returns to its initial length after removal of the mechanical constraint of stretching.

In particular, an elastomeric material complies with the definition of the standard D5538-98 of the ASTM relating to the standard use of thermoplastic elastomers.

Advantageously, the external diameter (20) of the annulus or portion of annulus at rest is comprised between 9.5 mm and 11.5 mm. The external diameter (20) of the annulus or portion of annulus at rest is preferably comprised between 10 mm and 11 mm and in a very preferred manner between 10.5 mm and 11.5 mm.

By external diameter (20) of the annulus (1) “at rest” according to the invention is meant the external diameter of the annulus after the aperture (12) of the envelope (1) has been filled with an incompressible fluid, without causing expansion of the envelope (1), as described above.

The external diameter (20) of the annulus defined above preferably has a value lower than the low values of the diameter-of the ciliary sulcus which it has been possible to measure in main Thus, the device according to the invention, the external diameter (20) of which can be increased under the effect of the pressure of the incompressible fluid introduced into the aperture (12), can be adapted to all patients and to all stages of development of an ocular condition linked to a diminution of the amplitude of accommodation of the eye, in particular presbyopia.

The elasticity characteristics of the biocompatible material constituting the envelope (1) are such that they make possible an expansion of the envelope (l) until an external diameter (20) of the annulus is attained which is included between 12 mm and 14 mm, is advantageously between 12 mm and 13.5 mm and is very much preferred between 12 nm and 13 mm.

According to a first embodiment, the closed tubular envelope (1) is a closed tubular annulus, as shown in FIG. 2.

According to a second embodiment, the closed tubular envelope (1) has the form of a portion of annulus, i.e. constitutes an open annulus, the aperture angle (221) is included between 1° and 100°, as shown in FIG. 3.

However, the embodiment in the form of the closed annulus should be preferred in order to avoid discontinuous contacts with the sulcus and the zonular fibres but have, on the contrary, a uniform contact applied over the whole surface of the annular groove of the posterior ciliary sulcus.

According to a first feature, the device according to the invention lacks specific means designed for filling or emptying the aperture (12). According to this particular feature of the device, the filling or emptying of the aperture (12) can be carried out by the introduction of a very small diameter tube through the envelope (1), for example the needle of a medical syringe. In this case, the elasticity of the polymeric material suffices to ensure the impermeability of the envelope (1) after withdrawal of the tube (needle), as is the case for example with a medical catheter.

According to a second particular feature of the device, the envelope (1) is equipped with a valve (13) making possible the entrance or exit of the incompressible fluid by the aperture (12).

The valve (13) may be of any known type.

FIG. 4 presents a first embodiment of a valve (13) constituted of a partial bevelled section of the envelope (1) making possible the introduction of a filling device for example the needle of a syringe. This type of valve is found conventionally in medical devices.

FIG. 5 illustrates a second embodiment of a valve (13) equipped with a mobile shutter the margins of which become contiguous under the effect of the pressure of the fluid within the aperture (12) and thus prevent the exit of the fluid. Such a type of valve is commonly designated by the term “Duckbill Valve”.

FIG. 6 illustrates a third embodiment of a valve (13), in which the aperture (12) of the envelope (1) is accessible through a tube of the catheter type, the extremity of which is equipped with a valve (13).

Advantageously, the diameter (21) of the envelope (1) at rest is included between 0.5 and 2 mm.

FIG. 7 shows an upper sagittal semi-section of an eye at rest without accommodation, with the device according to the invention in place in the ciliary sulcus.

FIG. 8 shows an upper sagittal semi-section of an eye at rest without accommodation with the device according to the invention in place in the ciliary sulcus, the eye having undergone an anterior ciliary sclerotomy opposite (121).

According to another aspect, the device according to the invention is characterized in that the thickness of the envelope (1) is at least 50 μm. The thickness of the envelope (1) may vary, in particular as a function of the nature of the biocompatible material used to produce this latter and the mechanical characteristics, in particular the elasticity modulus, of the said biocompatible material.

Depending on the material, the thickness of the envelope (1) is advantageously included between 50 μm and 1000 μn, and preferably between 100 μm and 500 μm.

Under the conditions of use, the device of the invention such as defined above, is characterized in that the aperture (12) of the envelope (1) is filled with an incompressible fluid.

The incompressible fluid may be of any kind compatible with long-term use in the organism. It is preferably a physiological solution currently used in the medical field, for example distilled water containing 0.09% by weight of dissolved sodium chloride or a BSS (buffered saline solution) type solution.

According to an additional technical advantage, the device according to the invention, which possesses great flexibility and reduced dimensions before filling, may be easily introduced into the ocular chamber in a folded form, for example by means of ocular microendoscopy, and may require only a small incision in the cornea prior to its implantation. The incompressible fluid may then be introduced into the aperture (12) of the envelope (1) of the device after implantation so that its definitive placement in the sulcus is attained.

Under the conditions of use, the device according to the invention is characterized in that the incompressible fluid is at a defined pressure for which the external diameter (20) of the annulus or portion of annulus compensates the loss of tension of the zonular fibres in a patient. In a very preferred manner, the defined pressure of the incompressible fluid is that for which the external diameter (20) of the annulus or portion of annulus restores tension in the zonular fibres.

In view of the anatomical dimensions of the ciliary sulcus, the device in place must be inserted in a diameter of about 2*R(sulcus) (with R=radius of the sulcus) and be capable of exerting an expansive force making possible an expansion of the radius of the scleral annulus of R(distended sulcus)-R(sulcus) of at least 400 μm/2 (because the maximal loss of amplitude of accommodation after 20 years of presbyopia corresponds to a distention of the cilio-equatorial diameter of 400 μm).

The external diameter (21) of the envelope (1) of the tubular device must not exceed 2 mm in order for it to be inserted in the anatomical sulcus. If the thickness of the biomaterial of the envelope (1) measures Ep (envelope), the volume of the aperture of the non-distended device can then be calculated according to the following formula:
V(aperture of the tube)=π[R(envelope)-Ep(envelope)]²×2π[R(sulcus)-R(envelope)].

For an envelope (1) of diameter (21) 2 mm and thickness 200 μm, the volume of the aperture (12) of the envelope at rest is 58.05 mm³, by application of the above formula.

Under the effect of the swelling pressure of the incompressible fluid in the aperture (12), the envelope (1) is distended with an increase of radius R(distended sulcus)-R(sulcus), which changes its sectional radius from R(envelope at rest) to R(distended envelope)=R(envelope)+[R(distended sulcus)-R(sulcus)].

The volume of the aperture (12) of the distended envelope (1) of the device has a final volume which can be calculated according to the following formula: V(distended aperture of the envelope)=π[R(envelope)+[R(distended sulcus)-R(sulcus)-Ep(envelope)]²×2λ[R(distended sulcus)-R(envelope)], namely 94.65 mm 3 if the reduction of the thickness of the wall of the tube under the effect of the expansion is neglected (Ep (envelope)=constant).

The defined pressure of the incompressible fluid for which the external diameter (20) of the annulus or portion of annulus brings about the compensation of the loss of tension of the zonular fibres in a patient can be easily determined by the specialist skilled in the art.

For example, the specialist skilled in the art may use a filling device such as previously mentioned which comprises a means for measuring the pressure of the incompressible fluid situated downstream from the filling device and upstream from the aperture (12) of the envelope (1). The external diameter (20) of the annulus or portion of annulus making possible the compensation of the loss of tension of the zonular fibres in a patient is attained when a sudden increase of the pressure of the incompressible fluid is measured.

With reference to FIG. 7, once in place in the ciliary sulcus and filled with incompressible fluid at the compensation pressure, the device of the invention ensures a surface tension which is transmitted from the surface of the external wall (10) over the anatomical quasi-circumference of the sulcus (22) and has for principal effect a removal of the ciliary body (23) from the geometric centre of the eye in the corresponding frontal plan, as well as a posterior tilting of the ciliary body (23) over its entire periphery with protrusion in to the peripheral anterior vitreous, leading to a placing of the zonular fibres under tension (24) which again become active for efficient accommodation. In the case of excessive sceral rigidity, the biomechanical effect of distention of the sceral annulus by the device in place in the sulcus according to the invention may be increased by making one or more incisions in the four quadrants according to the anterior ciliary sclerotomy procedure (25), as shown in FIG. 8.

The device according to the invention can also be used to diminish the intra-ocular pressure by increasing the degree of opening of the irido-corneal angle (26), which brings about an improvement of trabecular and uveo-scleral filtration.

The device according to the invention may also present anchorage surfaces situated on the external wall (10) or in the wall (1) making possible the delivery of any medicine by simple diffusion or active diffusion (medicarnentous reservoir) intended for the intermediate segment of the eye in particular to improve the efficacy of the device in presbyopia or to treat other ocular conditions (glaucoma, cyclitis, nitis, cataract).

The device according to the invention may also present attachment ones for the fixation of a precrystalline intraocular lens or another device for the 5 compensation of ocular refraction disorders (myopia, hypermetropia, astigmatism).

Thus, with the device of the invention, it is not necessary to carry out a measurement of the diameter of the sulcus of the patient to be treated, nor to carry out a measurement of the external diameter (20) of the annulus required to compensate the loss of tension of the zonular fibres in a patient, because the restoration of the tension in the zonular fibres can be determined, in particular by the measurement of the sudden change of pressure of the incompressible fluid within the aperture (12) of the envelope (1) when the external diameter (20) of the annulus has attained a value such that the device rests effectively on the sulcus and the zonular fibres and thus makes possible the reestablishment of a normal amplitude of accommodation.

In accordance with another feature, the device of, the invention is characterized in that the biocompatible elastomeric material is selected from a silicone, in particular a silicone rubber, a polyolefin homopolymer or copolymer, a polyurethane, a polyacrylic, a hydrogel, a mixture of hydrogel and silicone, a mixture of bovine collagen and hydrogel, a polyvinyl chloride elastomer, a. polytetrafluoroethylene (PTFE), a polysulfone or even a natural or synthetic rubber.

Biocompatible elastomers selected from the following compounds may in particular be used: polyurethane/methacryloyloxyethyl, phosphorylcholine, silicone-urethane, N-acyliminoethylene, diene/olefin, organopolysiloxanes and polydimethylsiloxanes, butadiene and acrylic thermoplastics, PVC/polyesters, PET, polyphosphazenes, latex, butylstyrene, fluoropolyurethane, nylon, SIS-/SBS-elastomers, polycarbonate-urethanes, polyether-etherketones, PTFE, PMMA, dexplastomers, alkylammonium-montmorillonite.

The elastomeric material called “Hytrel” consisting of a sequenced copolymer comprising the segments of the following formulae (1) and (3) may also be used:

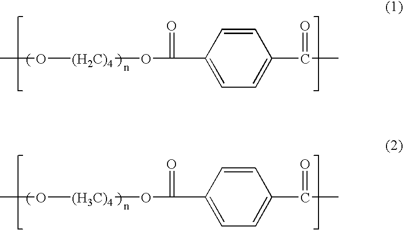

In which n is an integer included between 1 and 16.

The above block formula (1) is the ether of glycol and dimethyl terephthalate polytetramethylene (PTMEG/T), having a molecular weight included between 600 and 3000.

The block formula (2) is tetramethylene terephthalate (GT). The product “Hytrel” resulting from the condensation of the block formulae (1) and (2) above are sequenced copolymers constituted by crystalline 4GT hard segments and amorphous elastomeric soft segments of polyalkylene ether terephthalate.

By varying the proportions of the two phases according to the rules known to the specialist skilled in the art, the final characteristics of the final sequenced copolymer such as solidity, elasticity modulus, melting point, chemical resistance and permeability are determined. In particular, the higher the content of block CT, the harder the final copolymer. For the production and the characteristics of the copolymers of the “Hytrel” type, the specialist skilled in the art may advantageously refer to the monograph “Handbook of Thermoplastic Elastomers”, edited by Benjamin M. Walker and published in 1986.

For the use of a silicone elastomer designed for medical use, the specialist skilled in the art may advantageously refer to the standards “F 2038-00 e1” and “F 2042-00 e1” of the ASTM.

For the use of an elastomer of the olefin type, the specialist skilled in the art should advantageously refer to the standard “D-5593-99 e1” of the ASTM.

In general, for the production of the device according to the invention, the specialist skilled in the art should advantageously refer to the standards “F 1441-92 (1998)” and “F 2051-00” of the ASTM, relating to implantable devices of elastomeric material.

According to another advantageous characteristic, the device according to the invention comprises surface modifications of the envelope (1) useful as means for the impregnation of the envelope (1) by a composition containing an active ingredient or a combination of active ingredients of a medicine.

Once the device is placed in situ in the eye, the impregnated surface of the envelope (1) releases locally the active ingredient or combination of active ingredients, for example one or more actives ingredients of those conventionally used in ophthalmology.

According to yet another advantageous characteristic, the device of the invention also comprises a groove placed on the surface of the envelope (1), this groove defining an annulus in the internal diameter of the device, the said groove of the annulus consisting of a means for the fixation of a precrystalline intraocular lens adapted to the correction of various disorders of refraction associated with presbyopia for example, myopia, hypermetropia, associated or not with an astigmatism.

1. Device for treating an ocular condition linked to a diminution of the amplitude of accommodation of the eye due to a loss of tension of the zonular fibres caused by an increase of the diameter of the lens, said device being designed to be implanted surgically in the ciliary sulcus, behind the iris, and defined so as to compensate the loss of tension of the zonular fibres by exerting on the ciliary sulcus a pressure tending to increase its diameter, characterized in that the said device comprises a closed tubular envelope (1) constituted of an elastic material impermeable to fluids, the envelope (1) having the form of an annulus or portion of an annulus having an external diameter (20) predetermined at rest, the internal wall (11) of the envelope (1) defining an aperture (12) designed to be filled with an incompressible fluid tending to increase the external diameter (20) to a value at which the compensation for the loss of tension of the zonular fibres is obtained.

2. Device according to claim 1, characterized in that the envelope (1) is constituted of a biocompatible elastomeric material.

3. Device according to claim 1, characterized in that the external diameter (20) of the annulus or portion of annulus (1) at rest is included between 9.5 mm and 11.5 mm, advantageously between 10 mm and 11 mm.

4. Device according to claim 1, characterized in that the external diameter (20) of the annulus or portion of annulus (1) at rest is included between 10.5 mm and 11.5 mm.

5. Device according to claim 1, characterized in that the envelope (1) is equipped with a valve (13) making possible the entry and exit of the incompressible fluid by the aperture (12).

6. Device according to claim 1, characterized in that the diameter (21) of the envelope (1) at rest is included between 0.5 and 2 mm.

7. Device according to claim 1, characterized in that the thickness of the envelope (1) is at least 50 μm.

8. Device according to claim 1, characterized in that the aperture (12) of the envelope (1) is filled with an incompressible fluid.

9. Device according to claim 8, characterized in that the incompressible fluid is at a specific pressure for which the external diameter (20) of the annulus or portion of annulus is likely to compensate the loss of tension of the zonular fibres in a patient.

10. Device according to claim 2, characterized in that the biocompatible elastomeric material is selected from a silicone, a polyolefin, a polyurethane, a polyacrylic, a hydrogel and a silicone, a mixture of bovine collagen and a hydrogel, a polyvinyl chloride elastomer, a polytetrafluoroethylene (PTFE) and a polysulfone.

11. Device according to claim 2, characterized in that the biocompatible elastomeric material is selected from silicone rubbers, polyvinyl chloride, polyolefin homopolymers or copolymers, urethane-based elastomers, the natural or synthetic rubbers.

12. Device according to claim 1, characterized in that the envelope (1) comprises surface modifications as a means for the impregnation of the envelope (1) with an active ingredient or a combination of active ingredients of a medicine.

13. Device according to claim 1, characterized in that it comprises a groove placed on the surface of the envelope (1), and defining an annulus in the interior diameter of the device, the said groove of the annulus consisting of a means for fixing a precrystalline intraocular lens.

14. Device according to claim 2, characterized in that the envelope (1) is equipped with a valve (13) making possible the entry and exit of the incompressible fluid by the aperture (12).

15. Device according to claim 2, characterized in that the aperture (12) of the envelope (1) is filled with an incompressible fluid.
